(19) United States
(12) Patent Application Publication
(10) Pub. No.: US 2016/0120792 A1
(43) Pub. Date: May 5, 2016

(54) FORMULATIONS AND METHODS FOR IMPROVING SKIN CONDITIONS

(71) Applicants: Harunobu AMAGASF, Phoenix, AZ (US); FREELIFE INTERNATIONAL, INC., Phoenix, AZ (US)

(72) Inventor: Harunobu Amagase, Phoenix, AZ (US)

(73) Assignee: Freelife International, Inc., Phoenix, AZ (US)

(21) Appl. No.: 14/890,266

(22) PCT Filed: May 8, 2014
(86) PCT No.: PCT/US14/37326

§ 371 Date: Nov. 10, 2015

Related U.S. Application Data
(60) Provisional application No. 61/822,027, filed on May 10, 2013.

(51) Int. Cl. A61K 8/97; A61K 8/73; A61K 8/49; A61Q 19/00; A61K 8/34

(52) U.S. Cl. CPC A61K 8/97; A61Q 19/00; A61K 8/347; A61K 8/34; A61K 8/4946; A61K 8/73; A61K 2800/591

(57) ABSTRACT

The present invention discloses compositions and methods of using compositions for synergistically improving the skin conditions. The present compositions include Lycium plant in combination with other herbal and/or cosmetic ingredients, especially penetration-stimulating agents. The present compositions can also be used for anti-aging effects including moisturizing, reducing wrinkles and fine lines, promoting more youthful appearance, improving complexion, tone and texture of skin by increasing skin metabolic rate, and by means of preventing dehydration, antioxidant effects and by synergistically improving dermal fibroblast regulation.

### CROSS-REFERENCE TO RELATED APPLICATIONS

This application claims the benefit of U.S. Provisional Patent Application No. 61/822,027, filed May 10, 2013, which is incorporated herein by reference as if set forth in its entirety.

### STATEMENT REGARDING FEDERALLY SPONSORED RESEARCH OR DEVELOPMENT

Not Applicable

### FIELD OF THE INVENTION

The invention generally relates to compositions and methods of using compositions for synergistically increasing skin conditions. Specifically, the present invention relates to compositions containing Lycium plant extracts in combination with other herbal and/or cosmetic ingredients, especially penetration-stimulating agents. The present invention can be used for improving skin conditions and anti-aging effects including moisturizing, reducing wrinkles and fine lines and promoting a more youthful appearance. The present invention can also be used for improving complexion, tone and texture of skin by increasing skin metabolic rate, and by means of preventing dehydration, antioxidant effects and by synergistically improving dermal fibroblast regulation.

### BACKGROUND OF THE INVENTION

Lycium barbarum is a Solanaceous defoliated shrubbery that grows in China, Tibet and other parts of Asia and its fruits are 1-2 cm long, bright orange-red ellipsoid berries. A ripe fruit has been used in Asian countries as a traditional herbal medicine and functional food. Concentrated extracts and infusions prepared from the berries have a history of use as ingredients in various soft or alcoholic drinks that were marketed for their benefits to anti-aging, vision, kidney and liver functions. In support of these traditional properties, recent studies indicate that extracts from Lycium barbarum fruit and one of its active compounds, polysaccharides (LBP) possess a range of biological activities, including effects on skin protection, aging, neuroprotection, anti-fatigue/endurance, increased metabolism, glucose control in diabetics, glaucoma, anti-oxidant properties, immunomodulation, anti-tumor activity and cytoprotection.

Along with the growing number of studies, it was reported that LBP, particularly LBP-5 (or LbGp-5) had promoted the in vitro survival of human fibroblasts in sub-optimal conditions. There still remains an unmet need for improved rebuilding of the in vivo/ex vivo skin matrix by synergistically improving dermal fibroblast regulation with ingredients delivered topically and that maintain the skin in a moist, healthy state and maintain the proper skin barrier lipid balance [Zhao H, Alexeev A, Chang E, Greenburg G, and Bojanowski K. Lycium barbarum glycoconjugates: effect on human skin and cultured dermal fibroblasts. Phytomedicine, 12 (1-2): 131-7, 2005].

Lycium barbarum intake has also been reported to exert photoprotective effects by inducing endogenous cutaneous antioxidant haem oxygenase-1 (HO-1), and might therefore protect humans against actinic skin damage leading to cancers. A LBP-standardized Lycium barbarum fruit juice was administered to hairless mice in the drinking water. It was found that mice drinking 1%-10% Lycium barbarum juice were dose-dependently protected from sun-simulated ultraviolet (SSUV) radiation-induced systemic immunosuppression, measured as the contact hypersensitivity reaction. It was confirmed that the protection was an innate property of the goji berry, because it was not observed with any excipients of Lycium barbarum fruit juice or vitamin C, which is found in high concentration in Lycium barbarum berry. It was observed that 5% Lycium barbarum juice induced the expression of HO-1 mRNA in the mice skin. Furthermore, inhibition of HO enzyme activity by injecting mice with the substrate antagonist, SnPP, abrogated the photoimmune protection. Lycium barbarum offers skin protection against immune suppression and oxidative stress by SSUV radiation [Reeve V E, Allanson M, Arun S J, Domanski D and Painter N. Mice drinking goji berry juice (Lycium barbarum) are protected from UV radiation-induced skin damage via antioxidant pathways. Photochemical & Photobiological Sciences, 9: 601-607, 2010].

U.S. Pat. No. 7,807,625 discloses short bioactive peptides, palmitoyl hexapeptide combined with Lycium barbarum extract in cosmetic composition. However, this patent did not show actual effects in humans by any in vivo clinical studies. There were in vitro tissue or cell culture studies to show some possible effects, but they are not necessary to actually show significant positive effects on human skin. We thus compared this palmitoyl hexapeptide combined with Lycium barbarum extract and newly combined additional ingredients listed in Table 1.

It is an object of the invention to provide a cosmetic composition for synergistically improving the skin conditions. The ideal composition should have the capability of stimulating cell activities through preventing dehydration, rebuilding of the in vivo skin matrix by synergistically improving dermal fibroblast regulation with ingredients delivered topically, especially penetrated through surface skin. Beyond all these effects, the ideal composition would also function much more quickly than the conventional cosmetic compositions, which usually do not show the effects until 4 weeks later.

It is a further object of the invention to provide a cosmetic composition that will maintain the skin in a moist, healthy state and maintain the proper moisture content and skin barrier lipid balance without any greasy feeling.

### SUMMARY OF THE INVENTION

In one aspect, the present invention relates to a composition for synergistically improving the skin conditions. The composition in synergy for improving the skin conditions in a human subject comprises a Lycium plant extract, at least one emollient, at least one antioxidant, at least one antimicrobial, at least one humectant, at least one skin conditioner, at least one skin soothing agent, and at least one additive. In one specific embodiment of the present composition, the composition comprises a mixture of palmitoyl polypeptides. Preferably, the mixture of palmitoyl polypeptides comprises palmitoyl tri- and hepta-peptides.

In one preferred embodiment of the present composition, the emollient may comprise dimethicone, isododecane, polyisobutene and coco-caprylate/caprate. The antioxidant may comprise 3-O-ethyl ascorbic acid, tocopheryl acetate and L-ergothioneine. The antimicrobial may comprise bakuchiol. The humectant may comprise sodium hyaluronate and Saccharomyces lysate extract. The skin conditioner may comprise ectoin, caprylyl methicone, retinyl palmitate, a mixture of palmitoyl tri- and hepta-peptides, acetyl glucosamine, panthenol, caprylyl glycol, caffeine, and lactic acid/glycolic acid copolymer. The skin soothing agent may comprise bisabolol and allantoin. The additive may comprise polyacrylate and decyl glucoside.

In another embodiment of the present composition, the composition may further comprise purified or synthesized L. barbarum polysaccharides (LBP). In one specific embodiment of the present composition, the LBP comprises LBP-5.

In another aspect, the present invention relates to a method of synergistically improving skin conditions by topically applying any of the disclosed compositions.

Other objects, features and advantages of the present invention will become apparent after review of the specification, claims and drawings.

### DETAILED DESCRIPTION OF THE INVENTION

### I. In General

Before the present materials and methods are described, it is understood that this invention is not limited to the particular methodology, protocols, materials, and reagents described, as these may vary. It is also to be understood that the terminology used herein is for the purpose of describing particular embodiments only, and is not intended to limit the scope of the present invention which will be limited only by any later-filed non-provisional applications.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, and “having” can be used interchangeably.

As used herein, “about” means within 5% of a stated concentration range or within 5% of a stated time frame.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications and patents specifically mentioned herein are incorporated by reference for all purposes including describing and disclosing the chemicals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

### II. The Invention

The present invention is generally applied to humans. One may also wish to treat non-human mammals. For example, one may wish to treat companion animals, such as cats and dogs. One may also use the present invention for veterinary purpose. For example, one may wish to treat commercially important farm animals, such as cows, horses, pigs, rabbits, goats, and sheep.

The term “improve skin condition,” as used herein, refers to moisturizing, reducing wrinkles and fine lines, promoting a more youthful appearance, improving complexion, tone and texture of skin by increasing skin metabolic rate, and by means of preventing dehydration, antioxidant effects and by synergistically improving dermal fibroblast regulation. To improve skin conditions and anti-aging effects in humans, the present invention discloses a method of increasing efficacies and odor of cosmetic products containing Lycium (Lycium barbarum and related species) in a combination with other herbal and/or cosmetic ingredients, especially penetration-stimulating agents.

In some embodiments, the present invention discloses compositions and methods of using such methods for promoting bodily attractiveness, covering or masking the physical manifestations of a disorder or disease and modulating or alleviating wrinkling, photo-damage, unevenness and dryness in the skin of a mammal. The compositions may additionally regulate skin condition and signs of skin aging (all perceptible manifestations as well as any other macro or micro effects) by regulating visible and/or tactile discontinuities in skin texture, including fine lines, wrinkles, enlarged pores, roughness and other skin texture discontinuities associated with aged skin with reduced irritation and dryness.

The term “Lycium,” “Lycium barbarum” or “L. barbarum,” as used herein, refers to a solanaceous defoliated shrubbery and the fruit (goji) has been a commonly prescribed traditional medicine in Asian countries for over 2,500 years. Modern studies indicate that L. barbarum and its main active constituents, L. barbarum polysaccharides (LBP) possess a range of biological effects, such as significantly increasing metabolic rate and body weight reduction in mice and rats. The observed effects may also include significant clinical improvements in general well-being, including energy levels, sleep quality, glucose control in diabetics, glaucoma, antioxidant properties, anti-aging, neuroprotection, anti-fatigue/endurance, immunomodulation, anti-tumor activity and cytoprotection.

The Lycium plants in this invention are the plants belonging to the family of solanaceous defoliated shrubbery, such as Lycium barbarum and Lycium chinense. The most suitable plant for this invention is Lycium barbarum (known as goji, gouqizi, Fructus lycii, or wolfberry). The preferred portion for this invention is the fruit of this plant. Leaf, root or stem may also be utilized for this invention. These materials can be processed as juice or dried by processing and/or extraction methods commonly known in the art.

The term “emollients,” as used herein, refers to materials used for the prevention or relief of dryness, as well as for the protection of the skin. A wide variety of suitable emollients are known and may be used herein. Sagarin, Cosmetics, Science and Technology, 2nd Edition, Vol. 1, pp., 32-43 (1972), incorporated herein by reference contains numerous examples of suitable materials. In one embodiment, a composition of the present invention may comprise one or more emollients. In one embodiment, a composition of the present invention may comprise one or more emollients selected from a group consisting of dimethicone, isododecane, polyisobutene and coco-caprylate/caprate. In one preferred embodiment, a composition of the present invention may comprise all the emollients of dimethicone, isododecane, polyisobutene and coco-caprylate/caprate.

The term “antioxidant,” as used herein, refers to a molecule that inhibits the oxidation of other molecules. Oxidation is a chemical reaction that transfers electrons or hydrogen from a substance to an oxidizing agent. Oxidation reactions can produce free radicals. In turn, these radicals can start chain reactions. When the chain reaction occurs in a cell, it can cause damage or death to the cell. Antioxidants may terminate these chain reactions by removing free radical intermediates, and inhibit other oxidation reactions. Antioxidants may often comprise reducing agents such as thiols, ascorbic acid, or polyphenols. In one embodiment, a composition of the present invention may comprise one or more antioxidants. In one embodiment, a composition of the present invention may comprise one or more antioxidants selected from the group consisting of 3-O-ethyl ascorbic acid, tocopheryl acetate, and L-ergothioneine. In one preferred embodiment, a composition of the present invention may comprise all the antioxidants of 3-O-ethyl ascorbic acid, Tocopheryl acetate, and L-ergothioneine.

The term “antimicrobial,” as used herein, refers to an agent that kills microorganisms or inhibits their growth. Antimicrobial medicines may be grouped according to the microorganisms they act primarily against. For example, antibacterials (commonly known as antibiotics) may be used against bacteria and antifungals may be used against fungi. They may also be categorized according to their function. Antimicrobials that kill microbes may be called microbicidal; those that merely inhibit their growth may be called microbiostatic. Disinfectants such as bleach are non-selective antimicrobials. In one embodiment, a composition of the present invention may comprise at least one agent as an antimicrobial. In one preferred embodiment, a composition of the present invention may comprise bakuchiol as the antimicrobial.

The term “humectant,” as used herein, refers to a substance used to keep things moist. A humectant is the opposite of a dessicant, which is used to keep things dry. Humectants may be hygroscopic substances. A humectant may be a molecule with several hydrophilic groups, most often hydroxyl groups, but amines and carboxyl groups, sometimes esterified, can be used as well. A humectant may attract and retain the moisture in the air by absorption, allowing the water to be used by other substances or by plants. A humectant may differ from a dessicant which also attracts moisture but adsorbs it instead of absorbing it, i.e., condenses the water onto the surface as a layer of film. When used as a food additive, the humectant has the effect of keeping the foodstuff moist. Humectants may sometimes be used as a component of antistatic coatings for plastics. Humectants may also be found in many cosmetic products where moisturization is desired, including treatments such as moisturizing hair conditioners and also commonly used in body lotions.

Humectants may also be used in topical dosage forms to increase the solubility of the active ingredient, to elevate its skin penetration and increase its activity time. Humectants may also elevate the hydration of the skin to minimize the dehydrating effect of some active ingredients like corticoids. Examples of humectants may include propylene glycol (E1520) as well as hexylene glycol and butylene glycol, glyceryl triacetate (E1518), vinyl alcohol, neoagarobiose, sugar polyols such as glycerol, sorbitol (E420), xylitol and maltitol (E965), polymeric polyols like polydextrose (E1200), quillaia (E999), lactic acid, urea, glycerin, aloe vera gel, MP Diol, alpha hydroxy acids like lactic acid, and honey.

The term “subject,” as used herein, may be a mammal, such as a human, but may also be an animal, e.g., domestic animals (e.g., dogs, cats and the like), farm animals (e.g., cows, sheep, pigs, horses and the like) and laboratory animals (e.g., rats, mice, guinea pigs and the like).

The term “skin,” as used herein, refers to any part of the human or animal skin, including the whole surface thereof, hair and nails.

The term “treatment,” as used herein, refers to the topical administration of an effective amount of the present composition effective to ameliorate undesired symptoms associated with a skin disease, to prevent the manifestation of such symptoms before they occur, to slow down the progression of the disease, and to slow down the deterioration of symptoms. Treatment may also include the effect of enhancing the onset of remission period, slowing down the irreversible damage caused in the progressive chronic stage of the disease, delaying the onset of said progressive stage. Treatment may further include the effect of lessening the severity or cure the disease, improving survival rate or more rapid recovery, or preventing the disease form occurring or a combination of two or more of the above.

The “effective amount,” as used herein, refers to a therapeutically or cosmetically effective amount for purposes of improving skin condition, determined by such considerations as may be known in the art. The amount must be effective to achieve one or more of the above desired therapeutic or cosmetic effects, depending, inter alia, on the type and severity of the disease to be treated and the treatment regime. The effective amount may be typically determined in appropriately designed clinical trials (dose range studies) and the person versed in the art will know how to properly conduct such trials in order to determine the effective amount. As generally known, an effective amount may depend on a variety of factors, including the affinity of the ligand to the receptor, its distribution profile, a variety of pharmacological parameters such as half life on the skin, on undesired side effects, if any, on factors such as age and gender, etc.

The term “dimethicone,” or “polydimethylsiloxane,” or “PDMS,” as used herein, refers to a group of polymeric organosilicon compounds that are commonly referred to as silicones. PDMS is the most widely used silicon-based organic polymer, and is particularly known for its unusual rheological (or flow) properties. PDMS is optically clear, and, in general, inert, non-toxic, and non-flammable.

The term “isododecane,” as used herein, refers to a branched chain aliphatic hydrocarbon with 12 carbons. Isododecane may be used as a solvent. Isododecane may also be used as a fragrance ingredient, an emollient or a perfuming agent.

The term “3-O-ethyl ascorbic acid,” as used herein, refers to a vitamin C derivative consisting of an ethyl group bound to the third carbon position. 3-O-ethyl ascorbic acid may inhibit the polymerization arising due to the biological dihydroxyindole in vivo caused by ultraviolet rays, whereby darkening of the skin was completely prevented.

The term “palmitoyl polypeptide,” or “palmitoyl oligopeptide,” as used herein, refers to a short chain of multiple amino acids connected to palmitic acid. The examples of palmitoyl oligopeptides may include palmitoyl tripeptide, palmitoyl tetrapeptide, palmitoyl pentapeptide, palmitoyl hexapeptide, palmitoyl hexapeptide-3. The commercially available palmitoyl oligopeptide may include Palmitoyl Pentapeptide-4 (previously named Palmitoyl Pentapeptide-3, Matrixyl®), Pentapeptide-18 (Leuphasyl®), Pentapeptide-19, Acetyl Pentapeptide and Palmitoyl Hexapeptide-14 (previously named Palmitoyl Hexapeptide-6).

The term “tocopheryl acetate,” or “vitamin E acetate,” as used herein, refers to a common vitamin supplement with the molecular formula C₃₁H₅₂O₃ (for ‘a’ form). A tocopheryl acetate is an ester of acetic acid and tocopherol (vitamin E). It may be often used in dermatological products such as skin creams. Tocopheryl acetate is not oxidized and can penetrate through the skin to the living cells, where about 5% is converted to free tocopherol and provides beneficial antioxidant effects.

Tocopheryl acetate may be used as an alternative to tocopherol itself because the phenolic hydroxyl group is blocked, providing a less acidic product with a longer shelf life. It is believed that the acetate may be slowly hydrolyzed once it is absorbed into the skin, regenerating tocopherol and providing protection against the sun's ultraviolet rays.

The term “bakuchiol,” as used herein, refers to a meroterpene (a chemical compound having a partial terpenoid structure) in the class terpenophenol. Bakuchiol may also be called 4-[(1E,3S)-3-ethenyl-3,7-dimethylocta-1,6-dienyl]phenol. Bakuchiol may be isolated from P. corylifolia showing activity against numerous Grampositive and Gram-negative oral pathogens. Bakuchiol may be used to inhibit the growth of Streptococcus mutans under a range of sucrose concentrations, pH values and in the presence of organic acids in a temperature-dependent manner and also to inhibit the growth of cells adhered to a glass surface.

The term “sodium hyaluronate,” as used herein, refers to the sodium salt of hyaluronic acid, a glycosaminoglycan found in various connective, epithelial, and neural tissues. Sodium hyaluronate, a long-chain polymer containing repeating disaccharide units of Na-glucuronate-N-acetylglucosamine, occurs naturally on the corneal endothelium, bound to specific receptors for which it has a high affinity. The polyanionic form, commonly referred to as hyaluronan, is a visco-elastic polymer normally found in the aqueous and vitreous humour.

The term “N-acetylglucosamine,” or “N-acetyl-D-glucosamine” or “GlcNAc” or “NAG,” as used herein, refers to a monosaccharide derivative of glucose. It is an amide between glucosamine and acetic acid. It has a molecular formula of C₈H₁₅NO₆, a molar mass of 221.21 g/mol, and it is significant in several biological systems. NAG is part of a biopolymer in the bacterial cell wall, built from alternating units of GlcNAc and N-acetylmuramic acid (MurNAc), cross-linked with oligopeptides at the lactic acid residue of MurNAc. This layered structure is called peptidoglycan (formerly called murein). GlcNAc is the monomeric unit of the polymer chitin, which forms the outer coverings of insects and crustaceans. It is the main component of the cell walls of fungi, the radulas of mollusks, and the beaks of cephalopods. Polymerized with glucuronic acid, it forms hyaluronan.

The term “panthenol,” as used herein, refers to the alcohol analog of pantothenic acid (vitamin B₅). Panthenol is a provitamin of B₅. In organisms it is quickly oxidized to pantothenate. Panthenol is a highly viscous transparent liquid at room temperature, but salts of pantothenic acid (for example sodium pantothenate) are powders (typically white). Panthenol is soluble in water, alcohol and propylene glycol, soluble in ether and chloroform, and slightly soluble in glycerin.

Panthenol may have two enantiomers, D and L. Only D-panthenol (dexpanthenol) is biologically active, however both forms have moisturizing properties. For cosmetic use, panthenol comes either in D form, or as a racemic mixture of D and L (DL-panthenol). Panthenol's expanded chemical formula is HO—CH₂—C(CH₃)₂—CH(OH)—CONH—CH₂CH₂CH₂—OH.

The term “Saccharomyces lysate extract,” as used herein, refers to an extract from Saccharomyces lysate. Saccharomyces lysate is a group of fungi that ferment sugars. Saccharomyces lysate may be a source of beta-glucan, which is a good antioxidant. Live yeast-cell derivatives have been shown to stimulate wound healing, It has also been suggested that Saccharomyces lysate may have positive effects on inflammation, because of heat-shock proteins.

The term “allantoin,” as used herein, refers to a chemical compound with formula C₄H₆N₄O₃. It is a diureide of glyoxylic acid. Allantoin may also be called (2,5-Dioxo-4-imidazolidinyl) urea, 5-ureidohydantoin or glyoxyldiureide. Allantoin may be purified from botanical extracts of the comfrey plant or from the urine of most mammals. Allantoin may also be chemically synthesized.

The term “decyl glucoside,” as used herein, refers to the chemical compound of (2R,3R,4S,5S,6R)-2-Decoxy-6-(hydroxymethyl)tetrahydropyran-3,4,5-triol.

The term “retinyl palmitate” or “vitamin A palmitate,” as used herein, refers to the chemical compound of [(2E,4E,6E,8E)-3,7-Dimethyl-9-(2,6,6-trimethyl-1-cyclohexenyl)nona-2,4,6,8-tetraenyl]hexadecanoate. Retinyl palmitate is the ester of retinol (vitamin A) and palmitic acid, with formula C₃₆H₆₀O₂.

The term “ectoin,” as used herein, refers to a neutral non-ionic, strong water-binding, organic molecule of low molecular weight occurring in halophilic bacteria. These bacteria synthesize ectoin as a natural protection against extreme environmental conditions such as intense sun irradiation. The number of Langerhans cells decrease significantly in aged skin and hence the immuno-protective potential of the skin is reduced. This effect is more distinct in sun-unprotected skin. The topic application of Ectoin before sun exposure can prevent this UV-induced effect. Furthermore, ectoin shows singlet oxygen quenching properties and thus can prevent oxidative damage in skin. Ectoin also has a preventive effect on UV-A-induced large-scale mutations of the mitochondrial DNA in human dermal fibroblasts. It is strongly suggested that this property has an effect against skin wrinkle formation.

The term “ergothioneine,” as used herein, refers to the chemical compound of 3-(2-Sulfanylidene-1,3-dihydroimidazol-4-yl)-2-(trimethylazaniumyl)propanoate. Ergothioneine is a thiourea derivative of histidine, containing a sulfur atom in the imidazole ring. Ergothioneine may be a naturally occurring amino acid.

The term “bisabolol,” as used herein, refers to the chemical compound of 6-methyl-2-(4-methylcyclohex-3-en-1-yl)hept-5-en-2-ol. Bisabolol may be also known as levomenol, which is a natural monocyclic sesquiterpene alcohol.

The term “polyacrylate,” “acrylate polymer,” or “acrylics,” as used herein, refers to a group of polymers formed from acrylate monomers which includes acrylic acid or its derivatives.

The term “caffeine,” as used herein, refers to the chemical compound of 1,3,7-Trimethyl-1H-purine-2,6(3H,7H)-dione3,7-Dihydro-1,3,7-trimethyl-1H-purine-2,6-dione. Caffeine may also be found in varying quantities in seeds, leaves, and fruit of some plants, where it acts as a natural pesticide that paralyzes and kills certain insects feeding on the plants, as well as enhancing the reward memory of pollinators. Caffeine may be most commonly consumed by humans in infusions extracted from the seed of the coffee plant and the leaves of the tea bush, as well as from various foods and drinks containing products derived from the kola nut. Other sources of caffeine may include yerba maté, guarana berries, guayusa, and the yaupon holly.

In one embodiment, a composition of the present invention may comprise at least one humectant. In another embodiment, a composition of the present invention may comprise either sodium hyaluronate or Saccharomyces lysate extract. In one preferred embodiment, a composition of the present invention may comprise both sodium hyaluronate and Saccharomyces lysate extract.

The term “skin conditioner” or “skin conditioning agent,” as used herein, refers to a substance used to maintain or improve skin condition, e.g., enhancing the appearance of dry or damaged skin by reducing flaking and restoring suppleness. In one embodiment, a composition of the present invention may comprise at least one skin conditioner. In another embodiment, a composition of the present invention may comprise at least one skin conditioner selected from the group consisting of caprylyl methicone, retinyl palmitate, a mixture of palmitoyl tri- and hepta-peptides, ectoin, acetyl glucosamine, panthenol, caprylyl glycol, caffeine, and lactic acid/glycolic acid copolymer. In one preferred embodiment, a composition of the present invention may comprise all of the skin conditioners of caprylyl methicone, retinyl palmitate, a mixture of palmitoyl tri- and hepta-peptides, ectoin, acetyl glucosamine, panthenol, caprylyl glycol, caffeine, and lactic acid/glycolic acid copolymer.

The term “skin soothing agent,” as used herein, refers to a substance for treating sensitive or irritated skin by inhibiting inflammatory mediators. In one embodiment, a composition of the present invention may comprise at least one skin soothing agent. In another embodiment, a composition of the present invention may comprise either allantoin or bisabolol. In one preferred embodiment, a composition of the present invention may comprise both allantoin and bisabolol.

The term “additive,” as used herein, refers to a substance which is added to stabilize a composition of other substances. An additive may include a stabilizer, or a surfactant. Any suitable substance may be used as an additive. In one embodiment, a composition of present invention may comprise a polyacrylate as a stabilizer and decyl glucoside as a surfactant. A surfactant, also called a surface active agent, may include any ingredient that helps two substances that normally do not mix to become dissolved or dispersed in one another.

Herein, Applicants provide formulations and methods for increasing efficacies and fragrance of Lycium and related species by combining different preparations of Lycium extract, juice and freeze-dried materials with other herbal and cosmetic materials for the purpose of improving skin conditions in humans by increasing fibroblast generation, increasing metabolic rate, and by means of tonic, antioxidant effects.

In one embodiment of the present invention, a composition for synergistically improving skin conditions may comprise Lycium barbarum extract, at least one emollient, at least one antioxidant, at least one antimicrobial, at least one humectant, at least one skin conditioner, and at least one additive. In one embodiment, the Lycium barbarum extract may comprise fruit extract, or purified or synthesized LBP, e.g., LBP-5.

In one preferred embodiment, the composition may comprise a mixture of palmitoyl polypeptides. More preferably, the composition may comprise a mixture of palmitoyl tri- and hepta-peptides.

The composition for synergistically improving skin condition may be in any suitable forms. For example, the compositions and the medicament according to the invention may be applied topically, either as a gel, liquid, spray, and ointment, patch, as cotton gauze soaked with the composition according to the invention or as a power.

Applicants found that the interaction of multiple elements in the present composition produce an effect for improving skin condition greater than the sum of their individual effects of the elements. The following example demonstrates the synergistic effect for improving skin condition of the present compositions.

As one preferred embodiment, Applicants produced a composition by combining Lycium barbarum extract, e.g., fruit extract, or purified or synthesized LBP, e.g., LBP-5, with various cosmetic ingredients listed in Table 1 (Composition A). After topical administration of Composition A on the skin of a subject, the skin conditions were significantly improved as compared with those after topical administration of either Lycium barbarum extract plus palmitoyl hexapeptide (Composition B) or placebo control (Composition C) (Table 2).

Furthermore, Composition A containing Lycium barbarum fruit extract with various cosmetic ingredients listed in Table 1 shows unexpected, much better performance for improving skin conditions than either Composition B or Composition C. For example, the skin after topical administration of Composition A shows increasing feeling of healthier, more radiant, firmer, more hydrated/moisturized and younger looking skin, with improved complexion, tone and texture improvements, dark spot reduction and wrinkle reduction than those after topical administration of either Compositions B or C. Composition A shows these significant effects within 1-2 weeks. On the other hand, Composition B shows a slight improvement on the skin, but not statistically significant compared to the baseline level. Placebo control material of Composition C had no effect, or rather fell into negative effect territory (Table 2). Thus, Composition A has an unexpectedly much better effect of improving skin conditions in humans by improving skin conditions and anti-aging effects than (B) a composition including Lycium barbarum extract plus palmitoyl hexapeptide or (C) placebo control. The unexpected improved effect of Composition A also includes moisturizing, reducing wrinkles and fine lines, promoting more youthful appearance, improving complexion, tone and texture of skin by increasing skin metabolic rate, and by means of preventing dehydration, antioxidant effects and by synergistically improving dermal fibroblast regulation.

TABLE 1
List of ingredients in Composition A:

| | | | |
|---|---|---|---|
| LBP-5 Complex ™ (Water, | dimethicone | isododecane | 3-O-ethyl ascorbic acid |
| Lycium barbarum Fruit | | | |
| Extract) | | | |
| a mixture of palmitoyl tri- and | tocopheryl | Bakuchiol | sodium hyaluronate |
| hepta-peptides | acetate | | |
| acetyl glucosamine | panthenol | Saccharomyces | allantoin |
| | | lysate extract | |
| decyl glucoside | polyisobutene | caprylyl glycol | coco-caprylate/caprate |
| caprylyl methicone | retinyl | Ectoin | L-ergothioneine |
| | palmitate | | |
| bisabolol | polyacrylate | caffeine | lactic acid/glycolic acid |
| | | | copolymer. |

TABLE 2
The effect of Compositions A, B and C was investigated
by subjective questionnaire in a randomized, placebo-controlled,
blinded, cross-over human clinical trial. Each value indicates
percentage changes from baseline.

| | Change from baseline |
|---|---|
| Preparations | (in 2 weeks) |
| Composition A (Blend of Lycium extract plus | 66% |
| various ingredients listed in Table 1) | |
| Composition B (Lycium barbarum extract plus | 19% |
| palmitoyl hexapeptide) | |
| Composition C (Placebo Control with base | −26% |
| materials without any active ingredients) | |

The Lycium plants in this invention may include the plants belonging to the genus Lycium including approximately 70 species, such as Lycium barbarum, Lycium chinense, Lycium barbarum cv. ‘Tianjinense’, Lycium chinense var. potaninii and others. Lycium barbarum has various vernacular names, such as Chinese wolfberry, Barbary wolfberry, boxthorn, Chinese boxthorn, matrimony vine, Chinese matrimony vine, kuko (Japanese), red medlar, the Duke of Argyll's Tea Tree, gou qi (Mandarin), kei tze (Cantonese), gugija (Korean), c\dot{u} kh\dot{o}'i (Vietnamese), g\check{a}o gèe (Thai), and dretsherma (Tibetan). Its fruit is called Lycium Fruit, Fructus lycii (“fruit of Lycium”), lycii berries, lycii fructus, lycii fruit, dried wolfberries, gou qi zi (Chinese), gouqizi (Chinese), goji, Tibetan goji berry, goji berry, goji juice. Its dried root is called Di Gu Pi or Digupi. Other names are Bastard Jasmine, Box Throne, Common Matrimony Vine, Prickly Box, Tea Plant and Tea Tree (Bryan, et al. 2008; PDR, 2007). The preferred portion for the present invention may be the fruit of the plant. The other parts of the plant may also be utilized for this invention. These materials may be processed as juice, dried or freeze-dried by processing and/or extraction methods commonly known in the art.

In addition, the materials derived from the cell culture of the plants may also be utilized as materials for the present invention. The juice or extracts of Lycium plants in the present invention may be preferably made from plant materials by using water, alcohol, hydroalcoholic menstruums or supercritical fluid carbon dioxide as solvents. Prior to preparation or extraction, the Lycium plants may be squeezed or crushed with or without a moderate temperature to increase extraction efficiency, as is routinely understood in the art. It may also be possible to crush and homogenize the plants to make the juice after separating fruit, leaf, stem, root and other parts. The extract prepared for dietary intake may be utilized as it is, or in concentrated fluid or powder form after concentration under vacuum or by lyophilization.

Furthermore, the Lycium materials may be mixed with purified or synthesized LBP, such as LBP-5 or any other suitable polysaccharides. A suitable purified or synthesized LBP for the present invention may include those from any suitable sources, e.g., those derived from any other herbs. The Lycium materials may be extracted in any suitable solvents. A suitable solvent may include any polar protic solvents, e.g., water, nitromethane, acetic acid, alcohols, formic acid, any polar aprotic solvents, e.g., ethyl acetate, tetrahydrofuran, dichloromethane, acetone, acetonitrile, dimethylformate, dimethyl sulfoxide, any non-polar solvents, e.g., hexane, benzene, toluene, diethyl ether, chloroform, 1,4-dioxane, and others.

The administrative dosage for effectively improving skin conditions may depend on the ingredients as well as the age, body weight or body condition of the subject. In one preferred embodiment of the present invention, it is recommended to apply topically about 0.01-100 milliliters, preferably 0.1-50 milliliters, more preferably 0.5 milliliters at least twice daily (in the morning and again in the evening) by adults as a serum preparation of the combination of Lycium extract with dimethicone, isododecane, 3-O-ethyl ascorbic acid, caprylyl methicone, retinyl palmitate, a mixture of palmitoyl tri- and hepta-peptides, tocopheryl acetate, bakuchiol, sodium hyaluronate, ectoin, L-ergothioneine, acetyl glucosamine, panthenol, Saccharomyces lysate extract, allantoin, bisabolol, polyacrylate, decyl glucoside, polyisobutene, caprylyl glycol, coco-caprylate/caprate, caffeine and lactic acid/glycolic acid copolymer.

The dosage range for each ingredient may include, but not limited to, the following:
Lycium plant extract preparations: 0.001 microgram to 100,000 g per day. Any sources for Lycium plant extract may be applicable for the present invention, e.g., fruit, whole plant, leaf, stem, root or any other parts of the plant. Furthermore, any suitable extraction solvents may be used for preparation, such as water (cold, warm or hot), or any other suitable solvents alone or in combination of solvents having any suitable ratios.
Dimethicone: 0.001 nanogram to 100,000 g per day. Any suitable sources of dimethicone may be used for preparation, such as those naturally extracted from water (cold, warm or hot) or from any other suitable solvents. Dimethicone may be applied in any suitable solvents alone or in combination of solvents having any suitable ratios. Dimethicone may also be synthesized.
Isododecane: 0.001 nanogram to 100,000 g per day. Any kind of sources may be used for preparation, such as those naturally extracted from water (cold, warm or hot), or any other suitable solvents alone or in combination of solvents having any suitable ratios. Isododecane may also be synthesized.
3-O-ethyl ascorbic acid: 0.001 nanogram to 100,000 g per day. Any kind of sources may be used for preparation, such as those naturally extracted from water (cold, warm or hot) or any other suitable solvents alone or in combination of solvents having any suitable ratios. 3-O-ethyl ascorbic acid may also be synthesized.
Caprylyl methicone: 0.001 nanogram to 100,000 g per day. Any kind of sources may be used for preparation, such as those naturally extracted from water (cold, warm or hot), or any other suitable solvents alone or in combination of solvents having any suitable ratios. Caprylyl methicone may also be synthesized.
Retinyl palmitate: 0.001 nanogram to 100,000 g per day. Any kind of sources may be used for preparation, such as those naturally extracted from water (cold, warm or hot), or from any other suitable solvents alone or in combination of solvents having any suitable ratios. Retinyl palmitate may also be synthesized.
A mixture of palmitoyl tri- and hepta-peptides: 0.00001 nanogram to 100,000 g per day.
Tocopheryl acetate: 0.001 nanogram to 100,000 g per day. Any kind of sources may be used for preparation, such as those naturally extracted from water (cold, warm or hot), or from any other suitable solvents alone or in combination of solvents having any suitable ratios. Tocopheryl acetate may also be synthesized.
Bakuchiol: 0.0001 nanogram to 100,000 g per day. Any kind of sources may be used for preparation, such as those naturally extracted from water (cold, warm or hot), or from any other suitable solvents alone or in combination of solvents having any suitable ratios. Bakuchiol may also be synthesized.
Sodium hyaluronate: 0.001 nanogram to 100,000 g per day. Any kind of sources may be used for preparation, such as naturally extracted from water (cold, warm or hot), or from any other suitable solvents alone or in combination of solvents having any suitable ratios. Sodium hyaluronate may also be synthesized.
Ectoin: 0.0001 nanogram to 100,000 g per day. Any kind of sources may be used for preparation, such as those naturally extracted from water (cold, warm or hot), or from any other suitable solvents alone or in combination of solvents having any suitable ratios. Ectoin may also be synthesized.
L-ergothioneine: 0.001 nanogram to 100,000 g per day. Any kind of sources may be used for preparation, such as those naturally extracted from water (cold, warm or hot), or from any other suitable solvents alone or in combination of solvents having any suitable ratios. L-ergothioneine may also be synthesized.
Acetyl glucosamine: 0.001 nanogram to 100,000 g per day. Any kind of sources may be used for preparation, such as those naturally extracted from water (cold, warm or hot), or from any other suitable solvents alone or in combination of solvents having any suitable ratios. Acetyl glucosamine may also be synthesized.
Panthenol: 0.001 nanogram to 100,000 g per day. Any kind of sources may be used for preparation, such as those naturally extracted from water (cold, warm or hot), or from any other suitable solvents alone or in combination of solvents having any suitable ratios. Panthenol may also be synthesized.
Saccharomyces lysate extract: 0.001 nanogram to 100,000 g per day. Any kind of extraction may be used for preparation, such as those extracted from water (cold, warm or hot), or from any other suitable solvents alone or in combination of solvents having any suitable ratios.
Allantoin: 0.001 nanogram to 100,000 g per day. Any kind of sources may be used for preparation, such as those naturally extracted from water (cold, warm or hot), or from any other suitable solvents alone or in combination of solvents having any suitable ratios. Allantoin may also be synthesized.
Bisabolol: 0.001 nanogram to 100,000 g per day. Any kind of sources may be used for preparation, such as those naturally extracted from water (cold, warm or hot), or from any other suitable solvents alone or in combination of solvents having any suitable ratios. Bisabolol may also be synthesized.
Polyacrylate: 0.001 nanogram to 100,000 g per day. Any kind of sources may be used for preparation, such as those naturally extracted from water (cold, warm or hot), or from any other suitable solvents alone or in combination of solvents having any suitable ratios. Polyacrylate may also be synthesized.
Decyl glucoside: 0.001 nanogram to 100,000 g per day. Any kind of sources may be used for preparation, such as those naturally extracted from water (cold, warm or hot), or from any other suitable solvents alone or in combination of solvents having any suitable ratios. Decyl glucoside may also be synthesized.
Polyisobutene: 0.001 nanogram to 100,000 g per day. Any kind of sources may be used for preparation, such as those naturally extracted from water (cold, warm or hot), or from any other suitable solvents alone or in combination of solvents having any suitable ratios. Polyisobutene may also be synthesized.
Caprylyl glycol: 0.001 nanogram to 100,000 g per day. Any kind of sources may be used for preparation, such as those naturally extracted from water (cold, warm or hot), or from any other suitable solvents alone or in combination of solvents having any suitable ratios. Caprylyl glycol may also be synthesized.
Coco-caprylate/caprate: 0.001 nanogram to 100,000 g per day. Any kind of sources may be used for preparation, such as those naturally extracted from water (cold, warm or hot), or from any other suitable solvents alone or in combination of solvents having any suitable ratios. Coco-caprylate/caprate may also be synthesized.
Caffeine: 0.001 nanogram to 100,000 g per day. Any kind of sources may be used for preparation, such as those naturally extracted from water (cold, warm or hot), or from any other suitable solvents alone or in combination of solvents having any suitable ratios. Caffeine may also be synthesized.
Lactic acid/glycolic acid copolymer: 0.0001 nanogram to 100,000 g per day. Any kind of sources may be used for preparation, such as those naturally extracted from water (cold, warm or hot), or from any other suitable solvents alone or in combination of solvents having any suitable ratios.

In one embodiment of the present invention, the inactive ingredients would have a smaller dose range than the active ingredients. A person having ordinary skill in the art would appreciate the amount of each one of the ingredients to use. In another embodiment, it would be reasonable to add ±10% of the amount of each ingredient as appreciated by a person having ordinary skill in the art.

In another embodiment, the present composition may further comprise a cosmetically acceptable vehicle. By the term “cosmetically acceptable vehicle,” we mean any substances which may assist in the penetration of the present composition through skins of a human or a non-human mammal.

In one specific embodiment, the cosmetically acceptable vehicle for the present composition may be selected from the group consisting of crosspolymer gel, butylene glycol, cyclopentasiloxane, glycereth-26, isododecane, polysilicone-11, glycerin, hydroxypropyl cyclodextrin, decyl glucoside, Lavandula angustifolia (Lavender) Oil, acrylates/C10-30 alkyl acrylate crosspolymer, polyvinyl alcohol, hexylene glycol, polysorbate 20, disodium EDTA, arginine, dextran, phenoxyethanol and ethylhexylglycerin.

In an alternate preferred embodiment, the composition may further comprise an emulsion, a lotion, a spray, an aerosol, a powder, an ointment, a cream or a foam.

Further, any additional materials may be mixed together to improve the aroma, texture and/or skin feel of the above combinations by masking undesirable odors or other sensory aspects. For such purposes, any ordinary fragrances and additive agents may be used. Preferably, lavender oil and/or any other essential oils or compound fragrances may be added. Pleasant aroma is very important for continuous daily usage of these products to maintain healthy skin conditions and lifestyle.

The following examples describing materials and methodology are offered for illustrative purposes only, and are not intended to limit the scope of the present invention.

### III. Examples

### Example 1

### A Controlled Human Clinical Study (Table 2)

This example describes a human clinical study which examined the efficacy of topically applied product of Composition A, which includes a combination of Lycium extract with dimethicone, isododecane, 3-O-ethyl ascorbic acid, caprylyl methicone, retinyl palmitate, a mixture of palmitoyl tri- and hepta-peptides, tocopheryl acetate, bakuchiol, sodium hyaluronate, ectoin, L-ergothioneine, acetyl glucosamine, panthenol, Saccharomyces lysate extract, allantoin, bisabolol, polyacrylate, decyl glucoside, polyisobutene, caprylyl glycol, coco-caprylate/caprate, caffeine and lactic acid/glycolic acid copolymer. The results of Composition A were compared with that of Composition B including Lycium barbarum extract plus palmitoyl hexapeptide, and that of Composition C (a placebo control), which includes base materials without any active ingredients. The compositions were topically applied on facial skin in healthy human adults after daily usage of each preparation at morning and evening everyday for 2 weeks.

At shown in Table 2, an application of Composition B including Lycium barbarum extract plus palmitoyl hexapeptide-containing serum showed a slight improvement in facial skin condition having only a 19% increase from the baseline level. However, the facial skin conditions after a topical application of Composition A, a combination Lycium extract plus various herbs and cosmetic ingredients listed in Table 1, were significantly improved by 66% over than the baseline level, which was much higher than that from Composition B. On the other hand, a topical application of Composition C, which was a placebo control including base materials and lacking any active ingredients, did not improve facial skin conditions, but rather it worsened them by 26% negatively.

These results indicate that the synergistic effect of various herbs and cosmetic ingredients added to the Lycium extract in Composition A causes an unexpected significant increase in the effect as compared with Composition B.

Commercially available Lycium plant, such as goji berries and its extracts, has been known to have various health benefits, either by itself or combined with other ingredients. The Lycium plant's benefits for skin conditions have been shown in the previous U.S. patents (U.S. Pat. No. 8,354,099; U.S. Pat. No. 8,101,211; U.S. Pat. No. 8,097,581; U.S. Pat. No. 7,943,583; U.S. Pat. No. 7,807,625; U.S. Pat. No. 7,781,409; U.S. Pat. No. 7,776,915; U.S. Pat. No. 7,678,393; U.S. Pat. No. 7,348,034) and previous publications [Reeve V E, Allanson M, Arun S J, Domanski D and Painter N. Mice drinking goji berry juice (Lycium barbarum) are protected from UV radiation-induced skin damage via antioxidant pathways. Photochemical & Photobiological Sciences, 9: 601-607, 2010; Zhao H, Alexeev A, Chang E, Greenburg G, and Bojanowski K. Lycium barbarum glycoconjugates: effect on human skin and cultured dermal fibroblasts. Phytomedicine, 12 (1-2): 131-7, 2005].

However, Applicants have developed compositions and methods for using the compositions for synergistically increasing skin conditions with increased significant efficacies of health benefits beyond the existing compositions and methods, such as Lycium extract combined with palmitoyl hexapeptide. Specifically, the present composition includes a unique combination of various ingredients as listed in Table 1.

While commonly skin condition improvements are not usually observed until 4 weeks or more with continuous use, the above newly discovered effects of the present composition showed relatively quick effects within 1-2 weeks. Therefore, these skin condition improvements must be caused synergistically by the unique ingredient combination of the present application.

It is generally difficult or even impractical to apply impenetrable ingredients through stratum to reach epidermis with a sufficiently large amount of the various ingredients of the compositions. Therefore, combining Lycium extract with an ingredient which has capability to reach necessary area would be the key to improve skin conditions. This invention is based on this unique combination of Lycium extract and penetrable ingredients. By combining these ingredients, Applicants found unexpectedly better benefits on skin conditions, especially moisturizing, reducing wrinkles and fine lines than the existing combination of Lycium extract and palmitoyl hexapeptide.

Cosmetic products can often have overly strong aromas, especially for those products that contain chemicals or herbal materials with unpleasant odors. In addition, oils that are used in cosmetics can be comedogenic and can leave an unwelcome greasy feeling. In this invention, Applicants have developed a product that contains a unique combination of ingredients in a convenient, oil-free cosmetic dosage form with a light, pleasant and natural fragrance.

Other embodiments and uses of the invention will be apparent to those skilled in the art from consideration from the specification and practice of the invention disclosed herein. All references cited herein for any reason, including all journal citations and U.S./foreign patents and patent applications, are specifically and entirely incorporated herein by reference.

It is understood that the invention is not confined to the specific reagents, formulations, reaction conditions, etc., herein illustrated and described, but embraces such modified forms thereof as come within the scope of the following claims.

1) A composition for improving the skin conditions in a human subject comprising: a Lycium plant extract, at least one emollient, at least one antioxidant, at least one antimicrobial, at least one humectant, at least one skin conditioner, at least one skin soothing agent, and at least one additive.

2) The composition of claim 1, wherein the emollient comprises at least one ingredient from the group consisting of dimethicone, isododecane, polyisobutene and coco-caprylate/caprate.

3) The composition of claim 1, wherein the antioxidant comprises at least one ingredient from the group consisting of 3-O-ethyl ascorbic acid, tocopheryl acetate and L-ergothioneine.

4) The composition of claim 1, wherein the antimicrobial comprises bakuchiol.

5) The composition of claim 1, wherein the humectant comprises sodium hyaluronate and Saccharomyces lysate extract.

6) The composition of claim 1, wherein the skin conditioner comprises at least one ingredient from the group consisting of ectoin, caprylyl methicone, retinyl palmitate, a mixture of palmitoyl tri- and hepta-peptides, acetyl glucosamine, panthenol, caprylyl glycol, caffeine, and lactic acid/glycolic acid copolymer.

7) The composition of claim 1, wherein the skin soothing agent comprises bisabolol and allantoin.

8) The composition of claim 1, wherein the additive comprises polyacrylate and decyl glucoside.

9) The composition of claim 1, wherein the Lycium plant extract comprises the fruit extract of Lycium barbarum.

10) The composition of claim 1, wherein the composition further comprises purified or synthesized L. barbarum polysaccharides (LBP).

11) The composition of claim 10, wherein the LBP comprises LBP-5.

12) The composition of claim 1, wherein the composition further comprises a cosmetically acceptable vehicle.

13) The composition of claim 12, wherein the cosmetically acceptable vehicle is selected from a group consisting of crosspolymer gel, butylene glycol, cyclopentasiloxane, glycereth-26, isododecane, polysilicone-11, glycerin, hydroxypropyl cyclodextrin, decyl glucoside, Lavandula angustifolia (Lavender) Oil, acrylates/C10-30 alkyl acrylate crosspolymer, polyvinyl alcohol, hexylene glycol, polysorbate 20, disodium EDTA, arginine, dextran, phenoxyethanol and ethylhexylglycerin.

14) A method of improving the skin condition for a human subject comprises the step of topically administrating an effective amount of the composition of claim 1 to the skin.

15) The method of claim 14, wherein the effective amount of the compositions is 0.5 milliliters for at least twice per day.

16) The method of claim 14, wherein the Lycium plant extract comprises an amount of 0.001 microgram to 100,000 g per day of the extract of Lycium barbarum.
